(19) United States
(12) Patent Application Publication
(10) Pub. No.: US 2009/0155175 A1
(43) Pub. Date: Jun. 18, 2009

(54) SIMULTANEOUS TRANSLUMINAL CORONARY SINUS APPLICATION OF CELLS IN CONJUNCTION WITH DEVICE RESYNCHRONIZATION THERAPY

(76) Inventor: Eckhard Alt, Munich (DE)

Correspondence Address: HANLEY, FLIGHT & ZIMMERMAN, LLC, 150 S. WACKER DRIVE, SUITE 2100, CHICAGO, IL, 60606, US

(21) Appl. No.: 12/273,510

(22) Filed: Nov. 18, 2008

Related U.S. Application Data
(63) Continuation-in-part of application No. 12/255,550, filed on Oct. 21, 2008.
Application No. 12/255,550 is a continuation of application No. 10/955,403, filed on Sep. 30, 2004, now Pat. No. 7,452,532.
Application No. 10/955,403 is a continuation-in-part of application No. 09/968,739, filed on Sep. 30, 2001, now Pat. No. 6,805,860.

(51) Int. Cl. A61K 49/00; A61K 35/12; A61N 1/362; A61N 1/39; A61M 25/10

(52) U.S. Cl. 424/9.1; 424/93.7; 607/3; 604/522; 604/96.01

(57) ABSTRACT

Method and instrumentation for repairing tissue of a heart in a patient's body and implantation of biventricular stimulation. Adult stem cells that have the capability to repair tissue of the selected organ are recovered by harvesting from the patient's body from the same site as the implant of the device and in the same procedure simultaneously. The harvested stem cells are then intraluminally applied through the coronary venous circulation. During the time the stem cells are being applied to the targeted tissue downstream, the designated vessel is selectively occluded to increase concentration and pressure of the applied adult stem cells within that vessel. The implantation of a left ventricular stimulating electrode through the coronary sinus is done within the same operative procedure and connected to either a pacemaker capable of biventricular stimulation for resynchronization therapy or biventricular stimulation and additional defibrillation.

### RELATED APPLICATIONS

This application is a continuation-in-part of U.S. patent application Ser. No. 12/255,550, filed Oct. 21, 2008, which is a continuation of U.S. patent application Ser. No. 10/955,403, filed Sep. 30, 2004, now U.S. Pat. No. 7,452,532, which is a continuation-in-part of U.S. patent application Ser. No. 09/968,739, filed Sep. 30, 2001, now U.S. Pat. No. 6,805,860, each of which are incorporated herein by reference in their entireties.

### BACKGROUND

The present disclosure relates generally to transluminal application of therapeutic cells in conjunction with resynchronization therapy for improvement of myocardial function, and more particularly to balloon catheter protected transluminal application of multipotent cells for repair of the heart in patients that show signs of reduced cardiac pumping function and that would benefit from a so-called cardiac resynchronization therapy by means of biventricular stimulation with and without the addition option of defibrillation.

In principle, the human body has generally three types of cells. One type constitutes cells that continuously undergo replication and reproduction, such as dermal cells and intestinal epithelial cells, for example. These cells, which have a life as short as ten days, are replaced by the same cell type which is replicating continuously. A second type of cell is differentiated in the adult state but has the potential to undergo replication and the ability to reenter the cell cycle under certain conditions, an example being liver cells. The liver has the capacity to regrow and repair itself even if a tumor is excised and a major portion of the liver is removed. The third cell type comprises those cells that stop dividing after they have reached their adult stage, such as neuro cells and myocardial cells.

For this third cell type or group of cells, the number of cells in the body is determined shortly after birth. For example, myocardial cells stop dividing at about day ten after delivery, and for the rest of its life the human body has a fixed number of myocardial cells. Changes in myocardial function results from hypertrophy of cells and not by division and new cell growth.

Although the absence of cell division in myocardial cells is beneficial to prevent the occurrence of tumors—which practically never occur in the heart—it is detrimental with regard to local repair capacities. During an individual's lifetime, myocardial cells are subjected to various causes of damage that irreversibly lead to cell necrosis or apoptosis.

The primary reason for cell death in the myocardium is ischemic heart disease—in which the blood supply to the constantly beating heart is compromised through either arteriosclerotic build-up or acute occlusion of a vessel following a thrombus formation, generally characterized as myocardial infarction (MI). The ischemic tolerance of myocardial cells following the shut-off of the blood supply is in a range of three to six hours. After this time the overwhelming majority of cells undergoes cell death and is replaced by scar tissue.

Myocardial ischemia or infarction leads to irreversible loss of functional cardiac tissue with possible deterioration of pump function and death of the individual. It remains the leading cause of death in civilized countries. Occlusion of a coronary vessel leads to interruption of the blood supply of the dependent capillary system. After some three to six hours without nutrition and oxygen, cardiomyocytes die and undergo necrosis. An inflammation of the surrounding tissue occurs with invasion of inflammatory cells and phagocytosis of cell debris. A fibrotic scarring occurs, and the former contribution of this part of the heart to the contractile force is lost. The only way for the cardiac muscle to compensate for this kind of tissue loss is hypertrophy of the remaining cardiomyocytes (accumulation of cellular protein and contractile elements inside the cell), since the ability to replace dead heart tissue by means of hyperplasia (cell division of cardiomyocytes with formation of new cells) is a rather secondary mechanism.

Other means of myocardial cell alteration are the so-called cardiomyopathies, which represent various different influences of damage to myocardial cells. Endocrine, metabolic (alcohol) or infectious (virus myocarditis) agents lead to cell death, with a consequently reduced myocardial function as well as chronic pressure overload following chronic hypertension. The group of patients that suffer myocardial damage following cytostatic treatment for cancers such as breast or gastrointestinal or bone marrow cancers is increasing as well, attributable to cell necrosis and apoptosis from the cytostatic agents. There is also a large group of patients with so-called cardiomyopathies, often of unknown origin that present clinically with the same consequences: reduced myocardial function.

Heretofore, the only means for repair has been to provide an optimal perfusion through the coronary arteries using either interventional cardiology—such as PTCA (percutaneous transluminal coronary angioplasty), balloon angioplasty or stent implantation—or surgical revascularization with bypass operation. Stunned and hibernating myocardial cells, i.e., cells that survive on a low energy level but are not contributing to the myocardial pumping function, may recover. But for those cells which are already dead, no recovery has been achieved.

The current state of interventional cardiology is one of high standard. Progress in balloon material guide wires, guiding catheters and the interventional cardiologist's experience as well as the use of concomitant medication such as inhibition of platelet function, has greatly improved the everyday practice of cardiology. Nevertheless, an acute myocardial infarction remains an event that, even with optimal treatment today, leads to a loss of from 25 to 100% of the area at risk—i.e., the myocardium dependent on blood supply via the vessel that is blocked by an acute thrombus formation. A complete re-canalization by interventional means is feasible, but the ischemic tolerance of the myocardium is the limiting factor.

A recent article published in the New England Journal of Medicine (Schomig A. et al., ‘Coronary stenting plus platelet glycoprotein IIb/IIIa blockade compared with tissue plasminogen activator in acute myocardial infarction,’ N Engl J Med 2000; 343:385-391), for which the applicant herein was a clinical investigator, reports on a study of the myocardial salvage following re-canalization in patients with an acute myocardial infarction. The average time until admission to the hospital in these patients was 2.5 hours and complete recanalization was feasible after 215 minutes, roughly 3.5 hours. Nevertheless, only 57% of the myocardium at risk could be salvaged by re-canalization through interventional cardiology by means of a balloon and stent. When the group of patients was randomized to the classical thrombolytic therapy, which is the worldwide standard (with no interventional means), only 26% of the myocardium at risk could be salvaged. This means that even under optimal circumstances more than 40% of the myocardial cells are irreversibly lost.

With the knowledge that many patients arrive at a hospital at from 6 to 72 hours after the acute symptoms of vessel blockage by a thrombus, one can assume that the average loss of affected myocardial tissue is in a range of from 75 to 90% following an acute MI.

As noted above, cells can survive on a lower energy level, referred to as hibernating and stunning myocardium. As the collateral blood flow increases or re-canalization provides new blood supply they can recover their contractile function. The principle of myocardial reperfusion, limitation of infarct size, reduction of left ventricular dysfunction and their effect on survival were described by Braunwald (Braunwald E. et al., ‘Myocardial reperfusion, limitation of infarct size, reduction of left ventricular dysfunction, and improved survival: should the paradigm be expanded?,’ Circulation 1989; 79:441-4).

Annually, about five million Americans survive an acute myocardial infarction. Clearly then, loss of affected myocardial tissue is a problem of major clinical importance. Currently, repair is limited to hypertrophy of the remaining myocardium, and optimal medical treatment by a reduction in pre- and after-load as well as the optimal treatment of the ischemic balance by β-blockers, nitrates, calcium antagonist, and ACE inhibitors.

If it were feasible to replace the dead myocardium (scar tissue) by regrowing cells, or revitalize hibernating or stunning myocardium by the paracrine effect of injected cells with regenerative potential. A method to recover those cells is described in Coleman Stubbers patent applications, U.S. provisional application Nos. 61/005,267, filed Dec. 4, 2007, and 61/091,687, filed Aug. 25, 2008. Further mechanisms of the action of stem cells or cells with regenerative potential are described in Intracoronary Administration of Autologous Adipose Tissue-Derived Stem Cells Improves Left Ventricular Function, Perfusion, and Remodeling after Acute Myocardial Infarction; VEGF is Critical for Spontaneous Differentiation of Stem Cells into Cardiomyocytes; Liposome-Mediated Transfection with Extract from Neonatal Rat Cardiomyocytes Induces Transdifferentiation of Human Adipose-Derived Stem Cells into Cardiomyocytes; The Cardioprotective Effect of Mesenchymal Stem Cells is Mediated by IGF-I and VEGF; The Anti-Apoptotic Effect of IGF-1 on Tissue Resident Stem Cells is Mediated Via PI3-Kinase Dependent Secreted Frizzled Related Protein 2 (Sfrp2) Release; Electrophysiological Consequence of Adipose-Derived Stem Cell Transplantation in Infarcted Porcine Myocardium; Genetically Selected Stem Cells from Human Adipose Tissue Express Cardiac Markers; VEGF Receptor Flk-1 Plays an Important Role in C-Kit Expression in Adipose Tissue Derived Stem Cells; and Electrophysiological Properties of Human Adipose Tissue-Derived Stem Cells. Such a technique would have a profound impact on the quality of life of affected patients.

As noted earlier herein, in addition to ischemic heart disease other reasons exist for the reduction of myocardial cells that contribute to the pumping or electrical function of the heart. Among them are the cardiomyopathies, which describe a certain dysfunction of the heart. Reasons are many, such as chronic hypertension which ultimately leads to a loss in effective pumping cells, and chronic toxic noxious such as alcohol abuse or myocarditis primarily following a viral infection. Also, cell damage in conjunction with cytostatic drug treatment is becoming of greater clinical relevance. Not only the contracting myocardium becomes effected, but also the so called conduction system of the heart. Clinical symptoms are slow or too fast heart rates, generally called sinus node disease, AV Block conduction block and re-entry tachycardias and atrial flutter, atrial fibrillation, ventricular tachycardias and ventricular fibrillation are one feature of this disturbance. Another affects the intramyocardial conduction resulting in a limited synchronization of the pumping action of the right and left ventricle.

Many studies have proven the beneficial effect of a so-called cardiac resynchronization therapy by means of biventricular stimulation with and without additional provisions of a defibrillator (Chang S A et al., ‘Restoration of Left Ventricular Synchronous Contraction after Acute Myocardial Infarction by Stem Cell Therapy: New Insights into the Therapy for Acute Myocardial Infarction,’ Heart 2008; 94:995-1001).

Recently, several publications have shown the benefits of stem cell or multipotent cells on myocardial function (Abraham W T et al., ‘Cardiac Resynchronization in Chronic Heart Failure,’ N Engl J Med 2002; 346:1845-1853; Sutton M G et al., ‘Sustained Reverse Left Ventricular Structural Remodeling with Cardiac Resynchronization at One Year is a Function of Etiology: Quantitative Doppler Echocardiographic Evidence from the Multicenter InSync Randomized Clinical Evaluation (MIRACLE),’ Circulation 2006; 113:266-272) either in clinical or experimental studies. In summary, these studies showed local growth and survival primarily in the infarction border zone.

The application of stem or regenerative cell populations has been attempted by injection with small needles following an opening of the subject's chest and the pericardial sac. While a direct injection with small needles through the opening of the chest and the pericardial sac is certainly an option when additional cardiac surgical therapy is performed in which a thoracotomy is performed anyway, for most patients, this is not a primary option.

Also, reports have been published recently that employ a needle catheter that has been advanced through the aorta retrogradely into the left ventricle and small amounts of cells have been injected into the myocardium from the inside of the left ventricle. This therapy also has certain limitations such as cost associated with additional equipment and risks associated with sharp needles injected into the myocardium that can cause bleeding, arrythmias and an uneven distribution of cells. In addition, the puncture of an artery has the risk of local thrombus formation, unwanted peri- and post-interventional bleeding and damage to vascular structures including the aortic valve.

Recently, stem cells have shown they have a benefit on left ventricle synchronous contraction after acute myocardial infarction. The injection of stem cells has shown to resynchronize the ventricular contractility as described in the article by Chang S A et al., ‘Restoration of Left Ventricular Synchronous Contraction after Acute Myocardial Infarction by Stem Cell Therapy: New Insights into the Therapy for Acute Myocardial Infarction,’ Heart 2008; 94:995-1001. Similar experiences have been reported by our group in a recent study employing Intracoronary application of stem cells (Valina C et al., ‘Intracoronary Administration of Autologous Adipose Tissue-Derived Stem Cells Improves Left Ventricular Function, Perfusion, and Remodeling after Acute Myocardial Infarction,’ European Heart Journal 2007; 28:2667-2677).

Another form of improvement of left ventricular dyssynchrony is the in the meantime routinely applied resynchronization therapy with so-called biventricular synchronization. A recent Editorial even aims toward an implantable defibrillator in patients with heart failure in conjunction with a cardiac resynchronization therapy. This resynchronization therapy is especially useful in patients that develop a broad QIS complex during pacing and experience a New York Heart Association class III or IV heart failure. Life expectancy for those patients is low and these patients show worsening or even death within the subsequent two years of the diagnosis. Therefore the editorial aims toward a routine use of a defibrillator if a cardiac resynchronization therapy is considered in order to reduce the long-term morbidity and mortality. They claim two effects are responsible: the increased synchronization of the ventricle and therefore the higher efficiency in pumping of the myocardium and on top of this, the abolishment of arrhythmia episodes.

It is the aim of the current disclosure to provide an improved treatment for patients with heart failure that reduces operative interventional risks and costs with a maximum benefit for the patient's longterm outcome.

### SUMMARY

An example method of interventional medicine is described herein. The example method that includes an intraluminal application of cells that have the capability to replace necrotic tissue of a failing organ, such as the heart in the case of a MI, to allow resumption of myocardial function and, therefore, improve pumping performance of the myocardium in conjunction with the implantation of a biventricular stimulation device for cardiac resynchronization in the same operative/interventional procedure. The example procedure is oriented on the clinical practice of interventional cardiology following the principle that only those approaches that are both (a) relatively easy to perform, with little or no risk to the patient but a potentially high benefit, and (b) highly cost effective, are likely to be routinely applied in everyday medicine. An important aspect of at least some of the examples described is that the cells to be used in the intraluminal or transluminal application are autologous adult stem cells, which are derived from the same patient that has suffered the infarction. The cells are harvested and separated before injection, from the same individual (autologous transplantation) from which the cells were extracted. In a case of failing tissue of the myocardium, these cells are then injected into the coronary vasculature such as a coronary vein in a retrograde manner.

Furthermore, the stem cells need a certain contact time to adhere and migrate from the vascular bed into the infarcted myocardial area. In contrast to previous approaches, in which patches or applications through needles into the infarcted area have been considered, the approach of some of the example(s) described herein has shown that the most effective way to deliver the cells to the infarcted area is through the vascular tree of coronary arteries, arterioles and capillaries that supply the infarcted area. For injection into the venous system in a retrograde manner, an occlusion balloon over the wire type catheter is inflated inside the coronary sinus.

In addition, while the blood flow is still blocked, the stem cells are supplied by slow application through the balloon catheter over a relatively short period of time, on the order of 30 seconds to 5 minutes, for example. That is, the stem cells are injected through the inner lumen of the catheter while the balloon is inflated, and therefore, no washout occurs. Thus, this transvascular application of cells through the venous system of the heart during a period when flow or perfusion is ceased enables the cells to successfully attach to the vessel or myocardial wall for them to migrate to the tissue. And further, to overcome more actively the endothelial barrier following the increased pressure in the vascular bed which is attributable to the retrograde flow of cells being limited through the inflated balloon catheter.

Preferably, a steerable guiding catheter together with a balloon catheter or a steerable balloon catheter alone is employed for the intraluminal application, and the occlusion of the blood vessel is performed by inflating the balloon of the catheter for a time interval prescribed to increase the concentration of cells delivered to the site. Initially, over an introducer sheath of appropriate size or a direct venous cut down, a guide wire is advanced through the blood vessels into the vessel and then through vena subclavia and vena cava to the right atrium and thereafter the catheter is advanced over the guide wire until the distal end of the catheter reaches a selected point in the vicinity of the site for delivering the cells into the coronary venous vasculature.

The autologous adult cells may be harvested from the patient's own body, such as from the patient's bone marrow, adipose tissue, or may originate from lipoaspirate, as the source of the cells to be delivered to the site. Preferably, the harvesting is performed within a sufficiently short time interval immediately prior to delivery of the cells to the organ site to enhance the likelihood of successful organ tissue repair. In addition to using a balloon guided catheter of appropriate size in which the balloon size is expandable and fits the size of the coronary ostium or the coronary great vein or the other coronary venous branches and is adjustable from a size of 2-3 mm to more than 8 mm in order to tighten up the coronary sinus, the catheter preferably has an additional central lumen which extends through the whole length of the catheter and has features that make its distal part bendable. Preferably the last 20-10 cm of the catheter has a control to bend it in a certain angulation in order to canulate the coronary sinus more easily. This is especially important since the access to the coronary sinus in this case is selected not from the vena cava inferior and the groin, but from the upper part of the body through the vena cava superior. In order to engage the catheter in the coronary sinus, an upward direction is facilitated by means of additional use of a guide wire whose tip can also be bendable and advanced through the catheter. The possibility to shape the catheter in a U-shape, where the tip is controllable in the direction of up to 180 degrees, compared to the initial longitudinal axis of the catheter, enhances the success rate of canulation of the coronary sinus considerably. Both methods are considered to be applied: Either the guiding catheter is bendable and steerable and serves both as a long introducer stheathe that engages in the CS ostium through which a non-bendable balloon catheter is advanced and placed in the coronary sinus, or the balloon catheter is steerable and placed into the venous system through a conventional short introducer.

It is the aim of the present disclosure to provide apparatus and methods further to economically achieve the best treatment for patients with heart failure to minimize the cost of several interventional procedures, both as far as cost for operating rooms, anesthesia, and time spent in the OR, as well as increased logistic costs that can be saved if within the same procedure: a) the stem cells are harvested from the patient's own body; b) the stem cells are ready for injection during the same resynchronization procedure; c) the harvesting of the stem cells preferably occurs from the site where the future defibrillator is to be implanted; d) the same catheter that has canulated the coronary sinus serves for injection of the stem cells; e) is used to place a thin diameter, guide wire of preferably 14-18 thousandths of an inch thickness within the coronary sinus; f) to withdraw the balloon catheter and leaving the guide wire in place at the final destination in the coronary sinus for implantation of the left ventricular stimulation lead. After injection of the cells the balloon for stem cell injection or guiding catheter for cell injection is used for placement of the guide wire. The guide wire of which one end ends at the site of the future placement in the coronary sinus for the left ventricular simulation lead and the other end exits through the upper body part, preferably guided through the vena cephalica or subclavia of the right or left, preferably left shoulder, stays in place. Over this guide wire in place the coronary sinus left ventricular stimulation lead, which is either only a uni or bipolar electrode for low voltage stimulation of the heart, or incorporates an additional coil electrode which also comprises a defibrillation part with a coil and increased surface, is advanced into that location in the coronary vein adjacent to the left ventricle which has been determined to be the best site for active resynchronization. The defibrillator is then connected after implantation of other leads which stimulate the right ventricle and preferably the right atrium and have possibly electrode coil means for defibrillation, to the left ventricular lead in place.

In summary, one improvement consists of a one-time approach to recover cells, inject cells through the coronary sinus into the left ventricle to increase the cardiac function and to reduce the dyssynchrony of the ventricle by these stem cells and resynchronizing the heart in conjunction with an implantation of a biventricular stimulation in the same operative procedure. A further benefit is achieved if the cells for injection could be harvested during the same procedure, preferably from the same operative site which would reduce morbidity associated with the recovery of cells, since for the implantation of a defibrillator or pacemaker a cut into the skin and creation of a subcutaneous pocket is performed anyway. This site of the future implantation of the defibrillator could serve as the source for stem cells to be recovered after processing of the excised subcutaneous adipose tissue. In another embodiment of the disclosure, the recovery of adipose derived tissue resident stem cells from the patient's own body occurs at the time of the implantation of a pacemaker or defibrillator and the primary operative site is used to recover the tissue to obtain a cellular regenerative preparation from the excised tissue at the time of the implantation.

In this aspect of the disclosure, especially in patients in whom there is little subcutaneous adipose tissue, the injection of the cells occurs in a second procedure after the cells have been processed and grown to an adequate number that would suffice the needs for the number of cells to be injected in a second procedure. This injection could then take place, for example, during an otherwise coronary by-pass operation in which the patient's chest is open through an intracavitary needle catheter or through a second procedure with injection into the coronary sinus or the coronary arteries.

### BRIEF DESCRIPTION OF THE DRAWINGS

The above and still further aims, objectives, features, aspects and attendant advantages of the examples described herein will become apparent to those skilled in the art from the following detailed description of the examples described herein with reference to the accompanying figures.

FIG. 1 is a transparent front view of a patient showing example locations for obtaining autologous adult stem cells from the patient, and for injecting the harvested stem cells into the cardiovascular system and through a balloon catheter for introduction at the site of myocardial tissue damage to be repaired.

FIG. 2 is a detail view of the injection of cells at the designated site in FIG. 1.

FIG. 3 is a transparent front view of a patient illustrating an exemplary procedure for injecting harvested cells into the cerebral circulation of a patient

FIGS. 3A and 3B show respective syringes that operate the balloon or respectively, are used for injection of the cellular solution.

FIG. 3C is a view of a steerable balloon catheter with two lumina, one that runs from its proximal end to a balloon at the distal end and a second lumen which runs from proximal to distal either in parallel or in a coaxial way to the first lumen.

FIG. 4 depicts a simplified frontal view of a patient illustrating the site of implantation of the device, site of removal of subcutaneous adipose tissue, the venous internal axis to the right atrium, the heart with right and left ventricle septum and coronary sinus ostium.

FIG. 5 illustrates an example of the application of stem cells utilizing a steerable guiding catheter.

FIG. 6 depicts one embodiment of a rapid exchange type coronary sinus electrode.

### DETAILED DESCRIPTION

In an exemplary method to repair of myocardial tissue, the ischemically injured cardiac tissue is subjected to invasion by stem cells, preferably adult stem cells, with subsequent differentiation into beating cardiomyocytes which are mechanically and electrically linked to adjacent healthy host myocardium, thereby resembling newly formed and functionally active myocardium.

Until recently it had been hypothesized by most researchers that adult stem cells are tissue specific. It was thought that a certain stem cell-like population exists in every organ and is capable of differentiation into this certain tissue with exceptions to this rule regarding repair in heart and brain. Relatively recent studies have indicated an underestimated potential of these cells. It has been shown that murine and human neural stem cells (NSC) give rise to skeletal muscle after local injection (see, for example, Galli R et al., ‘Skeletal myogenic potential of human and mouse neural stem cells,’ Nat Neurosci 2000;3:986-991). Bone marrow stem cells have also been shown to replace heart tissue (cardiomyocytes, endothelium and vascular smooth muscle cells) after injection into lethally irradiated mice with a myocardial infarction (see Jackson K. A. et al., ‘Regeneration of ischemic cardiac muscle and vascular endothelium by adult stem cells,’ J Clin Invest 2001; 107(11):11395-402). The tissue damage in general appears to transmit signals which direct multi-potential stem cells to the site of destruction, and these precursors undergo a multi-step process of migration and differentiation at the organ site to replace damaged cells in form and function.

Experiments with cultured fetal cardiac myocytes or neonatal myocytes impose limitations owing to their heterologous nature and their possible induction of an immuno response necessitating an immuno-suppressive therapy. Complications and risks associated with an immuno-suppressant therapy are an increased susceptibility to infection and the possible development of malignancies. In addition, it is speculated that only a few patients would be willing to undergo a long term immuno-suppressive therapy with all its negative side effects.

An alternative approach by Prockop suggests that marrow stromal cells act as stem cells for non hematopoetic tissue and are capable to differentiate into various types of cells including bone, muscle, fat, hyaline cartilage and myocytes (Prockop D. J. et al., ‘Marrow stromal cells for non hematopoetic stem tissues,’ Science 1997; 276:71-74).

Some recent findings have stimulated interest in adult cardiomyocytes. A report in Nature describes the ability to inject adult bone marrow stem cells from transgenic mice into the border of infarcted myocardial tissue (Orlic D. et al., ‘Bone marrow cells regenerate infarcted myocardium,’ Nature 2001; 410:701-5). According to this report, these adult stem cells are capable of differentiation into cardiomyoblasts, smooth muscle cells and endothelial cells after injection. The infarcted myocardium implied that the transplanted cells responded to signals from the injured myocardium which promoted their migration, proliferation and differentiation within the necrotic area of the ventricular wall.

In addition, several recent publications have shown the benefit of injection of regenerative cells into the heart not only for improving myocardial function, but also to improve synchronization.

While studies have shown that the long term survival of patients with heart failure depends less on the clinical improvement and more on left ventricular reverse remodeling, more and more focus has been put on the cardiac resynchronization therapy. On the one hand it has been shown that cardiac resynchronization in fact has a benefit when two electrodes are implanted that stimulate the right and left ventricle. If the optimum delay between the timely events of stimulations is adapted to the patient, a much better efficiency of the cardiac pumping function is achieved since right and left ventricle are pumping at the same time, making the septum in between a firm wall of resistance. In cases of a dyssynchrony, most of the time the left ventricle follows the conduction of the right ventricle and therefore there is no synchronous contraction of the heart together but rather a swinging or wobbling.

Studies like MIRACLE or early studies published by Abraham in the New England Journal in 2002, have laid the foundation and proven the concept.

However, resynchronization therapy, especially when it is accompanied by the implantation of a defibrillator at the same time, is an invasive measure that by itself is accompanied by a certain mortality, eventual lack of success and considerable cost of several tens of thousands of dollars.

The current approach suggests utilizing available resources best in a way that at the time of the resynchronization operation, the procedure in the sterile setting is simultaneously used to additionally improve left ventricular function by injecting regenerative cells into the coronary sinus. This is done by the following steps of a) using the same vascular approach and b) catheter material that is used to visualize the coronary sinus such as a balloon catheter, by c) using the same intravascular access sheaths through which the balloon catheter, which engages in the coronary ostium, is advanced, to d) use the same guide wire and finally e) to obtain the stem cell preparation from the location where the cut into the body is already made in order to implant the defibrillator or the biventricular stimulation at that site.

By this means, two measures are taken at the same time to improve myocardial function, resynchronization by the electrical device and resynchronization through stem cells plus a improvement in myocardial function through the injection of stem cells as shown in several studies (Valina C et al., ‘Intracoronary Administration of Autologous Adipose Tissue-Derived Stem Cells Improves Left Ventricular Function, Perfusion, and Remodeling after Acute Myocardial Infarction,’ European Heart Journal 2007; 28:2667-2677).

One is then left to consider the most effective techniques to obtain adult stem cells. The classical way to recover stem cells is a bone marrow tap. The bone marrow contains a wide variety of hematopoetic and mesenchymal stem cells in addition to the T-lymphocytes, macrophages, granulocytes and erythrocytes. By incubation with monoclonal antibodies specific for the respective cell lineages and by sorting and removing with a biomagnet after incubation with magnetic beads and cell sorting with FACS (fluoroscopy activated cell sorting), a highly enriched cell line of bone marrow derived stem cells can be insulated, cultured and grown.

Aside from the classical approach of a bone marrow tap, several reports state that cells from human adipose tissue contain a large degree of mesenchymal stem cells capable of differentiating into different tissues in the presence of lineage specific induction factors including differentiation into myogenic cells (see for example Zuk P. A. et al., ‘Multilineage cells from human adipose tissue: Implications for cell-based therapies,’ Tiss Engin 2001; 7(2):211-28). The interesting approach in this research is that out of a lipoaspirate of 300 cm3 from the subcutaneous tissue, an average of 2-6*108 cells can be recovered. Even if one assumes that after processing of this liposuction tissue and separation and isolation of the cells, only roughly 10% of these cells might be early mesenchymal multipotent cells characterized by surface markers such as SSEA 4, CD 117, CD 146, CD 44, CD 90, CD 105 and CD 73 and express Oct-4, the remaining number of cells is quite sufficient to be used for the intraluminal or transluminal transplantation process described herein.

A benefit of this latter approach is that culturing and passaging of the stem cells are avoided. The cost of culturing and reinjecting cells are so that they prevent a widespread clinical usage and additionally certain lineages are lost in culture, dependent on the culture medium.

Presently, for ethical, immunological and feasibility reasons the applicant herein submits that transplantation of autologous adult stem cells (derived from the same individual that suffers the heart failure) is the most straightforward and practical approach to repair failing myocardium. The examples described herein promote invasion of ischemically injured cardiac tissue by stem cells that firmly attach and subsequently undergo differentiation into beating cardiomyocytes that are mechanically and electrically linked to adjacent healthy host myocardium. Adhesion of the injected stem cells and their migration beyond the endothelial barrier after injection into the coronary vein has been demonstrated by my research group and confirmed by observation after several days of frozen sections using light microscopy and, subsequently, electron microscopy. For evidence of the transition of stem cells into cardiomyocytes, markers introduced into the stem cells before they are reinjected into the myocardial tissue to be repaired provide evidence of cardiomyogenic differentiation.

This is done by a green fluorescence protein (GFP) used as a marker, with introduction into the stem cell genome by lentiviral gene transfer. Cells are identified after transplantation by fluorescence microscopy. In my lab, we also generated a construct that Luciferase is under the control of a cardiogenic gene allowing the presence of differentiating stem cells in the heart after injection.

Reference will now be made to the accompanying figures of drawing in describing an exemplary process. It should be noted at the outset that the figures are not intended to be to scale, nor to do more than serve as a visual aid to the description. In those figures representing the human body or body parts, certain components may be exaggerated relative to others for the sake of emphasis or clarity of the respective accompanying description. The autologous adult stem cells (more broadly, multipotent cells) are harvested in one of the known ways generally described above, such as by bone marrow tap or preferably from adipose tissue, for processing and possible cultivation.

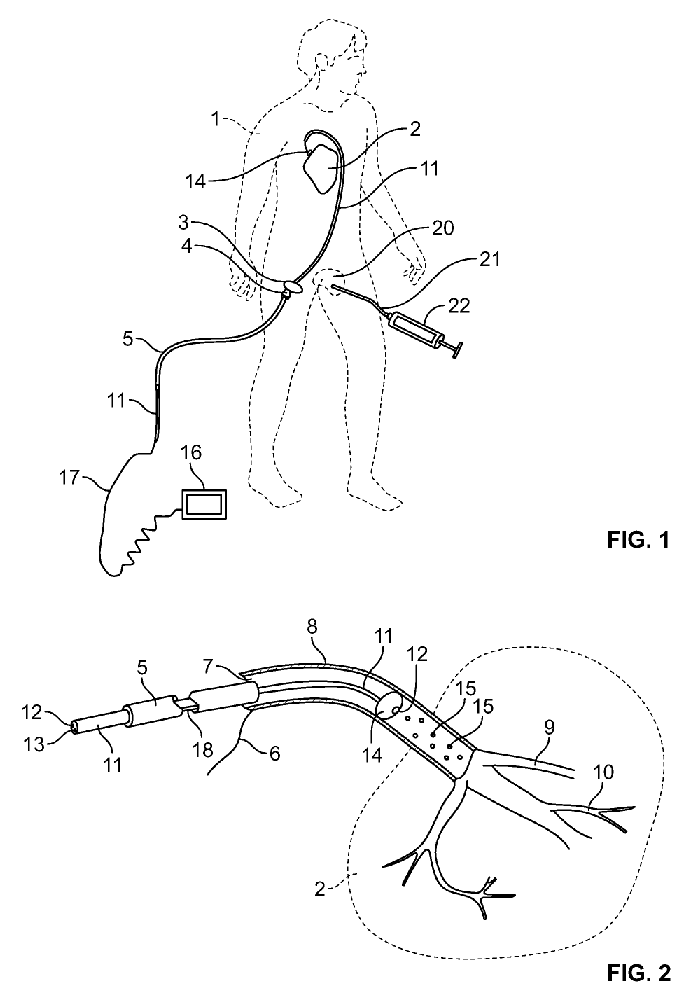

In an exemplary technique, and referring to FIG. 1, subcutaneous adipose tissue 20 is obtained from a liposuction procedure on the patient 1 during local anesthesia. In this procedure, a hollow canule or needle 21 is introduced into the subcutaneous space through a small (approximately about 1 cm) cut. By attaching gentle suction by a syringe 22 and moving the canule through the adipose compartment, fat tissue is mechanically disrupted and following the solution of normal saline and a vasoconstrictor epinephrine, a lipoaspirate of for example, about 30 to 300 cc is recovered (retrieved) within the syringe. The lipoaspirate is processed immediately according to established methods, washed extensively in phosphate buffered saline (PBS) solution and digested with for example, about 0.075% collagenase. The enzyme activity is neutralized with for example, Dulbecco's modified eagle medium (DMEM) containing for example, about 10% FBS (fetal bovine serum) and, following a centrifugation at for example, about 1200 G for about 10 minutes, a high density cellular pellet is obtained. Following filtration through an appropriately tight Nylon mesh in order to remove cellular debris, the cells are then ready to be injected into the area of concern in the patient's body or further processed and incubated overnight in a control medium of DMEM, FBS, containing an antibiotic, antimycotic solution. After the firm attachment of the stem cells to the plate, they are washed extensively with PBS solution to remove residual nonadherent red blood cells. Further cellular separation is conducted by separation with monoclonal antibodies coated on magnetic beads. Injection of the lipoaspirate is preferably performed as early as possible after recovery of the cells. A most elegant method of cell recovery is described in the patent application Coleman and Stubbers.

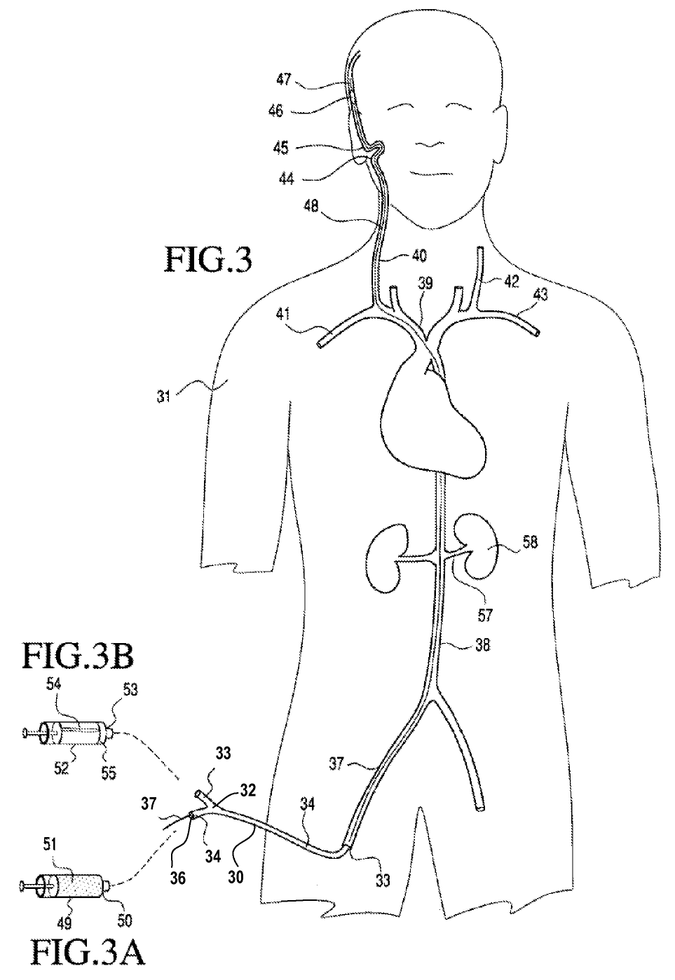

Referring to FIG. 2 as well as to FIG. 1, the recovered autologous adult regenerative cells are transplanted in the donor patient by intravascular or transvenous application for myocardial repair. This process is performed by first introducing a balloon catheter 11 into the cardiovascular system at the patient's groin 3 using an introducer 4, and through a guiding catheter 5 over a guide wire 18 into the aorta 6 and the orifice 7 of a coronary artery 8 of the heart 2 at or in the vicinity of the site where failed tissue owing to an infarction is to be repaired. The failed tissue is supplied with blood through artery 8 and its distal branches 9 and 10. The cells are hand injected or injected through the inner (central) lumen 12 of the balloon catheter 11 by means of a motor driven constant speed injection syringe 16 and connecting catheter 17 to entry point of the central lumen at the proximal end of catheter 11. The exit point of the central lumen 12 is at the distal end of catheter 11, which has been advanced into the coronary artery 8 in proximity to the site of the desired repair. The cells 15 are thereby delivered to this site by means of slow infusion over about 15-30 minutes, for example.

A problem encountered in attempting to do this resides in the fact that normally anything inside the blood vessel, including these cells, is separated from the parenchymatous organ or the tissue outside the vessel. In principle, blood flows through the larger arteries into the smaller arteries, into the arterials, into the capillaries, and then into the venous system back into the systemic circulation. Normally, the cells would be prevented from contacting the tissue to be repaired because of the endothelial lining and layer of the vessel that protects the tissue. However, under certain circumstances this barrier is overcome, and the cells can attach to the inside of the vessel, migrate and proliferate in the adjacent tissue. The increased pressure in the injection system promotes the injected cells to overcome the barrier.

In case of a more recent myocardial infarction, a possible endothelial ischemic damage owing to the infarction allows white blood cells, especially granulocytes and macrophages, to attach via integrins to the endothelial layer. The endothelial layer itself is dissolved in places by the release of hydrogen peroxide (H2O2) which originates from the granulocytes. This mechanism produces gaps in the endothelial layer that allow the stem cells to dock to the endothelial integrins and also to migrate through these gaps into the tissue to be repaired. An adjacent factor that enables the stem cells to migrate into the organ tissue is referred to as a stem cell factor that acts as a chemo-attractant to the cells.

One is then still faced with the problems of allowing enough of the repair cells to migrate into contact with the failing tissue and of achieving a high number of transplanted cells in the tissue. This is the principal reason for using a balloon catheter 11 or some other mechanism that will allow the physician (operator) to selectively block the antegrade flow of blood and the retrograde stem cell flow in case of a coronary artery injection and vice versa in case of an injection into the venous coronary sinus. A balloon catheter is preferred because it is a well known, often used and reliable device for introduction to a predetermined site in a vessel such as a pulmonary or coronary artery, to be used for angioplasty or wedge pressure control for example. In one of the described examples, the balloon 14 of catheter 11 is inflated with biocompatible fluid through a separate lumen 13 of catheter 11 to occlude coronary artery 8 and its distal branches 9 and 10, thereby causing perfusion through the vessel to cease. Inflation of the balloon may be commenced immediately before or at the time of injection of the stem cells through the inner lumen of the catheter, and is maintained throughout the period of injection. This enables the desired large number of adhesions of the cells 15 to the failing tissue to be achieved, because the absence of blood flow at the critical site of this tissue to be repaired has several advantageous effects. It prevents what would otherwise result in a retrograde loss of injected cells, an inability to increase the pressure at the injection site to overcome the endothelial barrier and to force the cells through the gap, and an antegrade dilution with blood flow of the cells being injected to that location through the catheter 11.

Dependent on the type and number of cells delivered, the blockage is maintained for a relatively short period of time, preferably on the order of about 30 seconds to about five minutes, in specific cases even longer, and in any event sufficient to allow a high concentration and considerable number of cell attachments to the tissue at the designated site, that will tend to guarantee a successful repair. A dosage of roughly 1.0 to 1.5 million cells per kg body weight of the patient has shown to be beneficial. This repair will extend as well to any failing tissue that may result from the blockage itself. In the case of a slow infusion of the cells, the period of blockage is maintained longer by steady inflation of the balloon over the injection period (e.g., up to about 30 minutes) for enhancement of contact and adherence of abmSC-P to the endothelium. The balloon is deflated, and the balloon catheter is removed from the patient after the procedure.

Previously reported studies have invariably employed a surgical approach for the application of the cells to be transplanted. Even if 5 to 10 sites of injection are performed with small needles, the complete inner, medial and outer layers of the myocardium are never covered. The examples described herein to use the natural distribution tree of the arterioles and the capillaries which is a more elegant solution, provided that (1) the transplanted cells can overcome the endothelial barrier and migrate into the tissue, (2) interventional cardiology means can restore blood flow into the infarcted area again, and (3) the cells have enough time to overcome the endothelial barrier.

In clinical practice there is a 96% success rate with interventional cardiology to restore blood flow following an acute myocardial infarction after an occlusion of a coronary artery. The experience that venously injected stem cells can be found in the myocardium, and the knowledge that in an acute myocardial infarction the endothelial barrier is considerably damaged (partly due to the H2O2 release of adhering neutrophilic cells), lead to the conclusion that a local injection into the infarcted area with an occlusive balloon to prevent a washout of the cells is the most desirable approach. The applicant herein has performed studies in the past with a technique called ‘BOILER’-lysis, in situations where older venous bypass grafts are occluded by a thrombus that has grown over a prolonged period of time. It was observed that an acute injection of a thrombolytic agent rarely dissolved these old thrombi. But after an over the wire balloon catheter was inserted into the occluded graft, a prolonged application of a thrombolytic substance such as urokinase was successful in achieving thrombolysis. The agent is injected at the tip of the balloon catheter, and is forced antegradely into the thrombus. The inflated balloon prevents a washout by the normal coronary circulation and allows the injection by a motor pump at a defined volume per time. For stem cell therapy and repair as performed by the example methods described herein, an injection over a period of about 1 to about 30 minutes is feasible and gives the cells sufficient contact time to adhere to the surface of the damaged endothelium.

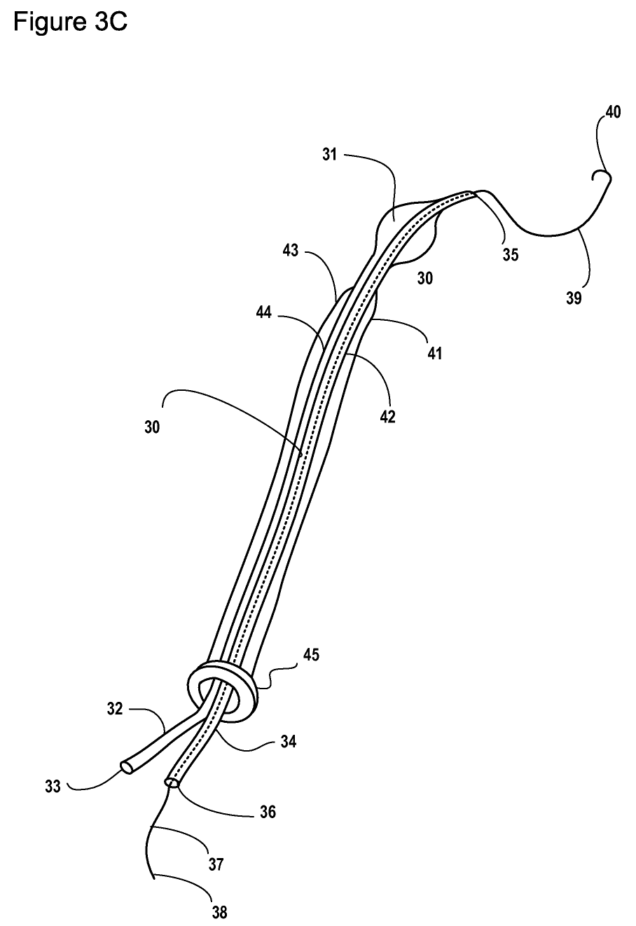

FIGS. 3, 3A, 3B, and 3C are diagrams useful to describe an example of a method for delivery of stem cells through a balloon-guided catheter to the venous system of the patient's heart. FIG. 3C depicts a dual-lumen catheter 30 with a balloon 31 on its distal end. Lumen 32 connects the balloon 31 with the proximal outlet 33. A second lumen 34 connects the distal end 35 with the proximal end 36 outside the body. This lumen is suitable to accommodate a guide wire 37 which runs from its proximal end 38 to its distal end 39 in the vascular system. The very distal end 40 even has the ability to be bended over an instrument and thereby pre-shaped. In addition the catheter has mechanisms 41, 42, 43 and 44 to change as the primarily straight catheter into a distally bended form via a mechanism 45. By turning mechanism 45, different traction and release to a mechanism 41-44 is applied which affects bending at the distal part of the catheter preferably at a distance of the last 20 cm of this catheter. By that way the catheter can be advanced in an initially straight way to accommodate a straight vessel, but in order to get engaged in the coronary sinus, the distal part can be bent up to a total U-shape where the tip 35 points in the direction toward the proximal end 33 and 36. The catheter 30 can be advanced over wire 37 to a more distal location close to 39 while the bended tip of the wire can be advances through the coronary sinus to a specific location desired. After advancing the catheter into the coronary sinus, the inflation of balloon 31 can tighten the lumen of the coronary venous vessel completely, and an injection through the inner of channel 34 with stem cells or regenerative cells exists then at distal opening 35. For that purpose the guide wire 37 is temporarily withdrawn. After injection of the cells, guide wire 37 is advanced again through the lumen 34 into its distal position; following this, the balloon catheter 30 is removed totally over the proximal end 38. Length 38 is long enough to accommodate the full length of catheter 30 by that way the wire 37 is kept in place and the distal tip 40 stays at the location where it has been brought before.

FIG. 3A depicts a syringe 49 with the distal conus 50 that can be connected to the proximal port 36 of catheter 30. The syringe contains a solution with stem cells 51 that can be injected through the lumen 36 of the catheter after removal of the guide wire 37. Syringe 52 has a distal connector 53. This syringe is connected to the proximal port 33 of lumen 32 that operates a balloon with an injection of either air (which is feasible in the venous system since a rupture of the balloon would only cause air embolism in the pulmonary artery) or the balloon could also be filled with some contrast dye. By inflating and deflating syringe 52 the size of the balloon 31 can be adapted to be either empty and nearly flat with the catheter diameter or being inflated to match the size of the venous vessel in the coronary sinus and tighten the vessel in a way that injection through the central lumen would increase the pressure in the distal venous segment in which the balloon is located.

In addition of using syringe 49 for injection of stem cells, the syringe can be used to inject contrast dye to visualize the coronary sinus with contrast in order to select the appropriate location of the tip 35 of the balloon catheter.

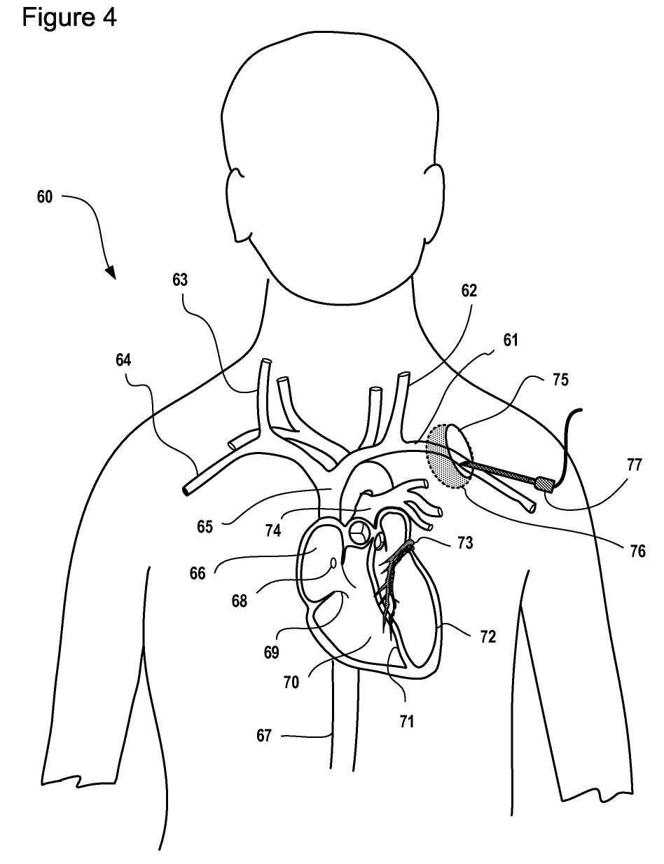

FIG. 4 shows a patient 60. The anatomy of the venous circulation of the upper limb is shown with vena subclavia respectively vena cephalica 61, jugular vein 62 on the left side, jugular vein 63 on the right side of the patient and subclavian cephalic vein 64 on the left side of the patient. Those veins gather into vena cava superior 65 which ends in the right atrium 66 together with vena cava inferior 67. The coronary sinus ostium is located in the right atrium at 68 close and above the tricuspid valve 69 which separates the right atrium from the right ventricle 70. The cavum of the right ventricle further is confined by septum 71 and the boundaries of the left ventricle are depicted 72. Coronary sinus 68 then divides into several branches which follow and accompany the arterial course of the arteries such as the interventricular vein 73 which accompanies the left anterior descending artery 72. The right ventricle empties into the pulmonary circulation 74. In addition to the anatomy also the incision at the site of the left shoulder 75 is shown, this approximate two inch long incision exposes a subcutaneous site where a defibrillator or a biventricular pacemaker is implanted at site 76. Through the same cut also venous access into either cephalic vein 61 or its continuations to the venous circulation is achieved by either a direct cut into the vein itself or by puncture by means of a Seldinger technique and the use of a hemostatic introducer. Over this introducer, the catheter 30 is initially advanced through the vena cava superior 65 and then located in the right atrium 66 and advanced into the coronary ostium 68. Further manipulation with the guide wire 37 and advancing its distal end 39 and the bended end 40 the wire and the balloon catheter is placed at the correct location to implant the cells by injection and to implant the defibrillator, respectively pacemaker electrode in a distal coronary venous vessel such as 73. In order to visualize the anatomy of the venous circulation distal from the coronary sinus 68, an injection of contrast dye through the balloon catheter lumen 34 can be done while the catheter is in place and the balloon preferably is inflated. Fluoroscopy and line angiography with x-rays depict the anatomy of the coronary venous system. This helps to make the right decision at which location the wire should be advanced and the balloon placed. Echocardiographic measurements during that procedure additionally can indicate which area of the left ventricle has the greatest delay and the greatest dyssynchrony in order to fine tune the decisions where to inject cells and where to place the electrode for left ventricular pacemaker stimulation.

After advancing the balloon catheter through the cut 75 and the introducer 77 into the venous access or as mentioned before through a direct cut down, the guide wire 37 left in place and sticking out from either the introducer which is a peel-away introducer 77 or through the direct cut-down of the venous access side.

Through cut down 75, the incision is used to remove part of the tissue, especially adipose tissue from the pocket 76 to make room for the future implant and at the same time to use the adipose tissue to recover valuable regenerative cells that can be used for injection into the heart or other sites to be treated within the body. The preparation of a regenerative cell population takes roughly 45-60 minutes, so during the operation the first step is to drape the patient with sterile coverage, to disinfect the skin at the area of incision 75, to make the incision and to recover the adipose tissue by preparation of the subcutaneous pocket between skin and fascia of the pectoralis muscle. The adipose tissue is removed and processed while the rest of the procedure continues such as canulation and finding of a venous access through which then balloon catheter 30 will be advanced into the coronary sinus. The first diagnostic means then is to shoot a radiographic depiction of the anatomy of the coronary sinus by means of injecting contrast dye via syringe 49 connected with its conus to the connector 36 of lumen 34. During the injection of the contrast dye, balloon 31 also is inflated in order to give a sufficient filling of all venous vessels. In addition, the time until the cells are prepared is used to perform an electrophysiology analysis of the underlying dyssynchrony as far as the electrical signals are concerned and a functional analysis by means of a transthoratic or a transesophageal echocardiography. Also, an injection of contrast dye into the pulmonary circulation and the filling of the left ventricle are feasible by means of advanced CT imaging, which can reconstruct the 3-dimensional picture of the left and right atria and ventricle following a contrast dye injection. Then guide wire 37 is advanced to the adequate position within the coronary sinus and distal by means of turning the wire with its bended end 40 in the right direction in order to get a selective location of the guide wire.

In the next step then the cellular preparation is injected into the coronary venous system after inflation of balloon 31 at the appropriate location in the coronary venous circulation and the balloon is occluded for the time of injection and thereafter in order to allow the stem cells to attach to the wall and overcome the venous endothelial layers by increased pressure in the system. The inflation of balloon 31 is important to create this down-stream pressure from opening 35 within the circulation when the cellular preparation 51 is injected by means of syringe 49. While the previous examples are given with the combination of a steerable balloon catheter placed in the ostium of the coronary sinus and or further advanced into the coronary veins, in another exemplary embodiment it is also understood, that the guiding catheter that canulates the ostium is steerable and that a non-steerable balloon catheter is placed through the lumen of the steerable guiding catheter in the coronary vein.

After the successful injection of the stem cells, balloon catheter 30 is removed and wire 37 is easily exchanged for a wire of smaller diameter before the balloon is removed or wire 37 matches the physical requirements of electrode 80 and its rapid exchange guidance 81.

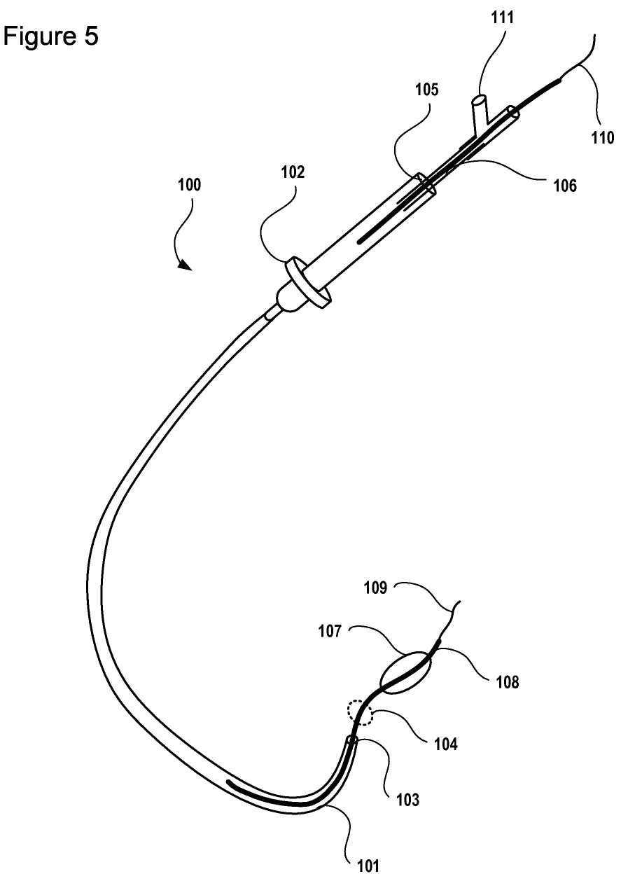

In another example of the application of stem cells illustrated in FIG. 5, a steerable guiding catheter 100 is used. The catheter 100 can be bended at its distal end 101 by a mechanism 102 which operates in a similar manner to the mechanism 45. The mechanism 102 activates the bending of the distal path of the balloon catheter through the mechanism 41-44. The example catheter 100 is a guiding catheter as known the art. At the distal tip, the mechanism 102 facilitates the insertion of the distal tip 103 of the catheter into the coronary sinus ostium 104. Through the proximal end of a lumen 105 a straight and/or pre-bent, but non-steerable balloon catheter 106, is advanced. At its distal end the balloon catheter 106 has a balloon 107 that can be inflated through a second lumen 111. A distal tip 108 of the balloon catheter 106 opens a distal lumen through which a guide wire 109 at its distal part and 110 at a proximal part of the guide wire can be applied. The distal part 108 of the balloon catheter 106 is located in the coronary sinus and the balloon 107 is able to occlude a coronary vein at side distal to the injection in the coronary sinus of the stem cells through the central lumen 108. An opening of the second lumen 111 is connected to the inflation of the balloon while the central lumen 108 is able to run from the proximal to the distal end and after the installation of the guide wire 110 to be used for the injection of stem cells.

In contrast to the previous disclosure in this case the guiding catheter is inserted into a vein and the steerable mechanism is dislocated while the balloon itself is applied without the steerable mechanism.

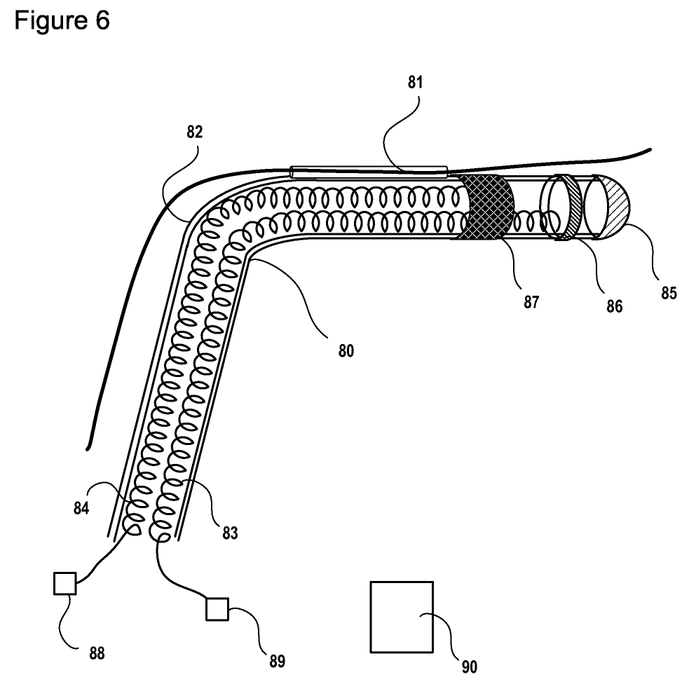

In FIG. 6, such an electrode is schematically shown with an internal insulation 82, an internal wire 83, or in case of two electrical contactors 83 and 84, which each connect to a distal electrode 85 that either at the tip or in form of a ring 86 or in form of a larger surface for defibrillation 87 connected to one of the proximal wires that have a connector 88 or 89 that make contact to a defibrillator or stimulator 90 which has a capability to accommodate several electrodes and defibrillator leads in the heart. In another embodiment of an over the wire type lead, the guide wire extends through a central lumen of the electrophysiology pacing and or defibrillator catheter and extends through a central distal opening. After retraction of the wire, the lumen has a self sealing valve like closing in order to prevent blood from entering.

After connecting to the appropriate wires, biventricular pacemaker or stimulator 90 with or without the capacity to defibrillate will be implanted in pocket 76 where the adipose tissue has been removed and the incision 75 will be closed.

In an alternative embodiment, autologous adult cells are harvested from the patient's own body, from the bone marrow, from adipose tissue, or from lipoaspirate at other locations as the source of the cells to be delivered to the target site at time of the biventricular device implantation and within the same operative setting. The harvesting should be performed within a sufficiently short time interval immediately prior to the time the cells are to be delivered, so as to enhance the likelihood of successful organ tissue repair.

Although certain methods, apparatus, and articles of manufacture have been described herein, the scope of coverage of this patent is not limited thereto. To the contrary, this patent covers all methods, apparatus, and articles of manufacture fairly falling within the scope of the appended claims either literally or under the doctrine of equivalents.

I claim:

1. A method for repairing tissue in a heart and improving the cardiac function, comprising:
delivering adult stem cells to the tissue of the heart to be repaired, by an intraluminal application through a venous coronary vessel; and
implanting a left ventricular electrode through a coronary sinus in the same operational procedure as injecting the stem cells.

2. A method as defined in claim 1, further comprising occluding the venous coronary vessel distal to the location of stem cell cell entry via the intraluminal application during at least a portion of the duration of the cell delivery to increase the concentration of cells delivered into the venous coronary vessel.

3. A method as defined in claim 1, further comprising harvesting subcutaneous adipose tissue derived stem cells through the same incision that serves for the implantation of the left ventricular electrode.

4. A method as defined in claim 1, further comprising imaging the coronary sinus.

5. A method as defined in claim 1, further comprising a steerable balloon guided catheter for placement in the coronary sinus and adapted to perform the delivery of the adult stem cells, the occluding of the blood vessel, and the implantation of a guide wire at a distinct location in the coronary sinus to facilitates the implantation of the left ventricular electrode.

6. A method of treating dyssynchrony in a patient by implanting a device for ventricular resynchronization and injecting stem cells into the heart within the same operative procedure, comprising:
injecting stem cells in a retrograde manner through at least one of a coronary sinus or a coronary venus circulation;
using a steerable balloon guided catheter to facilitate placement of a lead of the device for ventricular resynchronization; and
using the steerable balloon guided catheter for injecting the stem cells.

7. A method as defined in claim 6, further comprising recovering stem cells from the patient's body from its subcutaneous tissue within the same operative procedure.

8. A method as defined in claim 6, wherein the device for ventricular resynchronization is a cardiac stimulator.

9. A method as defined in claim 6, wherein the device for ventricular resynchronization is a cardiac defibrillator.

10. A method as defined in claim 6, wherein the device for ventricular resynchronization is a biventricular stimulator.

11. A balloon guided catheter comprising:
a first lumen that operates a distal balloon; and
a second lumen that runs from a proximal end to the distal end of the catheter,
wherein the balloon is bendable at the distal end of the catheter to accommodate the canulation of the coronary sinus.

12. A balloon guided catheter as defined in claim 11, wherein the catheter is at least approximately 50 cm in length.

13. A balloon guided catheter as defined in claim 11, wherein the balloon guided catheter is advanced through the venous system of a patient's body from the upper body.

14. A balloon guided catheter as defined in claim 11, wherein the balloon is bendable along at least approximately 20 cm of the balloon's distal end.

15. A balloon guided catheter as defined in claim 11, wherein the balloon defines a central lumen, and wherein an injection is introducible through the central lumen.

16. A balloon guided catheter as defined in claim 15, wherein the injection includes a contrast agent to aid in the visualization of a coronary venous anatomy.

17. A balloon guided catheter as defined in claim 15, wherein the injection is a cellular preparation of stem cells.

18. A balloon guided catheter as defined in claim 11, wherein the balloon guided catheter is advanceable over a guide wire.

19. A balloon guided catheter as defined in claim 11, wherein the balloon guided catheter implants a guide wire to position an electrode in the heart.

20. A method of treatment of heart failure in a patient comprising placing a steerable guiding catheter in the coronary sinus or coronary vein of the patient; injecting a regenerative cellular preparation through the guiding catheter into the coronary sinus circulation in a retrograde manner; and
using the same guiding catheter within the same operational procedure for implantation of a left ventricular stimulation electrode.

21. A method as defined in claim 20, further comprising injecting regenerative cells through a non-steerable balloon catheter placed through and within the guiding catheter.

22. A method as defined in claim 20, wherein the balloon is inflated during cell injection.

23. A method for repairing tissue in an organ comprising:
recovering a regenerative cellular preparation from adipose tissue of the body of a patient through an operation access; and
delivering the regenerative cellular preparation to the tissue of the organ to be repaired, by an intraluminal application through a vessel of the organ.
